(19) United States
(12) Patent Application Publication
(10) Pub. No.: US 2024/0404684 A1
(43) Pub. Date: Dec. 5, 2024

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Yudai YAMAZAKI, Nasushiobara (JP); Yusuke KANO, Edinburgh (GB); Sho SASAKI, Utsunomiya (JP)

(73) Assignee: CANON MEDICAL SYSTEMS CORPORATION, Otawara-shi (JP)

(21) Appl. No.: 18/678,151

(22) Filed: May 30, 2024

(30) Foreign Application Priority Data
Jun. 1, 2023 (JP) 2023-090788

(51) Int. Cl. G16H 40/20; G16H 20/00

(52) U.S. Cl. CPC G16H 40/20; G16H 20/00

(57) ABSTRACT

A medical information processing device using an artificial intelligence model of an embodiment includes processing circuitry. The processing circuitry acquires first data indicating a state of a patient before intervention of a medical procedure related to a predetermined disease, and second data indicating a state of the patient after intervention of the medical procedure. The processing circuitry calculates a reward on the basis of at least a variation in the first data and a variation in the second data. The processing circuitry causes the artificial intelligence model to learn policies for the medical procedure on the basis of the reward. The processing circuitry updates the artificial intelligence model through a learning of the policies.

### CROSS-REFERENCE TO RELATED APPLICATION

Priority is claimed on Japanese Patent Application No. 2023-090788, filed Jun. 1, 2023, the content of which is incorporated herein by reference.

### FIELD

Embodiments disclosed in this specification and drawings relate to a medical information processing device, a medical information processing method, and a storage medium.

### BACKGROUND

It is possible to provide high-quality medical care to patients using a quality control method in medical care, called clinical pathways. However, if a patient's disease is not a standard or representative disease, it may not be possible to apply clinical pathways to the patient. In particular, patients with cancer or cardiovascular disease have many complications, making it difficult to apply clinical pathways. Furthermore, in general medical departments, selection of a treatment or examination depends on the skill or experience of a doctor. Therefore, there is a need for a system for supporting selection of a treatment or examination suitable for a patient.

### BRIEF DESCRIPTION OF THE DRAWINGS

FIG. 1 is a diagram showing a configuration example of a medical information processing device in an embodiment.

FIG. 2 is a flowchart showing a series of processing flows of processing circuitry according to an embodiment.

FIG. 3 is a diagram showing a method of estimating the probability density function of unobserved medical data.

FIG. 4 is a diagram showing a method of estimating the probability density function of unobserved medical data.

FIG. 5 is a diagram comparing medical data before and after intervention of treatment which is a medical procedure.

FIG. 6 is a diagram comparing medical data before and after intervention of examination which is a medical procedure.

FIG. 7 is a diagram comparing medical data before and after therapeutic intervention which is a medical procedure.

FIG. 8 is a flowchart showing a series of processing flows of processing circuitry according to an embodiment.

FIG. 9 is a diagram showing an example of a display screen on which information based on a medical procedure to be performed on a target patient is displayed.

FIG. 10 is a diagram showing an example of a display screen on which information based on a medical procedure to be performed on a target patient is displayed.

### DETAILED DESCRIPTION

Hereinafter, a medical information processing device, a medical information processing method, and a storage medium according to embodiments will be described with reference to the drawings.

A medical information processing device using an artificial intelligence model of an embodiment includes processing circuitry. The processing circuitry acquires first data indicating a state of a patient before intervention (performance) of a medical procedure related to a predetermined disease, and second data indicating a state of the patient after intervention of the medical procedure. The processing circuitry calculates a reward on the basis of at least a variation in the first data and a variation in the second data. The processing circuitry causes the artificial intelligence model to learn policies for the medical procedure on the basis of the reward. The processing circuitry updates the artificial intelligence model through a learning of the policies. Accordingly, it is possible to recommend a medical procedure for intervention on the patient, such as a treatment or examination.

### [Configuration of Medical Information Processing Device]

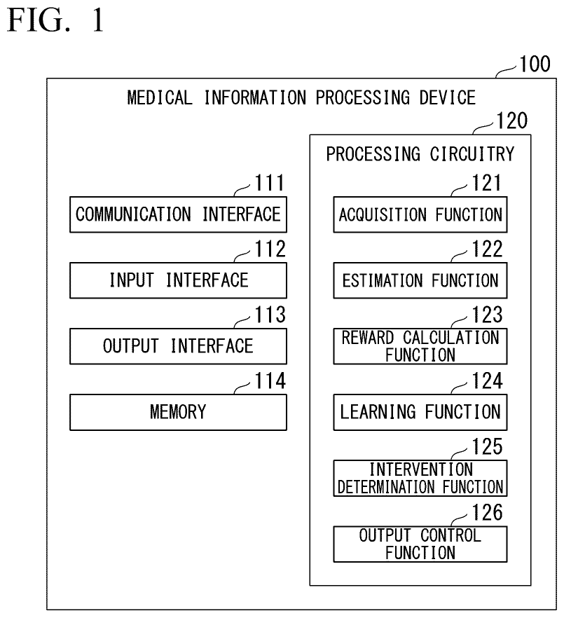

FIG. 1 is a diagram showing a configuration example of a medical information processing device 100 in an embodiment. The medical information processing device 100 includes, for example, a communication interface 111, an input interface 112, an output interface 113, a memory 114, and processing circuitry 120.

The communication interface 111 communicates with external devices via a communication network NW. The communication network NW may mean any information communication network using telecommunications technology. For example, the communication network NW includes a wireless/wired LAN such as a hospital backbone local area network (LAN), an Internet network, a telephone communication line network, an optical fiber communication network, a cable communication network, a satellite communication network, and the like. The communication interface 111 includes, for example, a network interface card (NIC), an antenna for wireless communication, and the like.

The input interface 112 receives various input operations from an operator, converts the received input operations into electrical signals, and outputs the electrical signals to the processing circuitry 120. For example, the input interface 112 includes a mouse, a keyboard, a trackball, a switch, a button, a joystick, a touch panel, and the like. The input interface 112 may be, for example, a user interface that receives voice input, such as a microphone. When the input interface 112 is a touch panel, the input interface 112 may also have a display function of a display 113a included in the output interface 113 which will be described later.

In this specification, the input interface 112 is not limited to one that includes physical operation parts such as a mouse and a keyboard. For example, examples of the input interface 112 also include electrical signal processing circuitry that receives an electrical signal corresponding to an input operation from an external input apparatus provided separately from the device and outputs this electrical signal to control circuitry.

The output interface 113 includes, for example, the display 113a and a speaker 113b. The display 113a displays various types of information. For example, the display 113a displays images generated by the processing circuitry 120, a graphical user interface (GUI) for receiving various input operations from an operator, and the like. For example, the display 113a is a liquid crystal display (LCD), a cathode ray tube (CRT) display, an organic electroluminescence (EL) display, or the like. The speaker 113b outputs information input from the processing circuitry 120 as audio.

The memory 114 is realized by, for example, a semiconductor memory element such as a random-access memory (RAM) or a flash memory, a hard disk, or an optical disc. These non-transitory storage media may be realized by other storage devices connected via the communication network NW, such as a network attached storage (NAS) or an external storage server device. Further, the memory 114 may include a non-transitory storage medium such as a read only memory (ROM) or a register. The memory 114 stores programs executed by a hardware processor of the processing circuitry 120, various calculation results of the processing circuitry 120, model information, and the like.

The model information is information (a program or an algorithm) that defines a conversion model MDL1, a reinforcement learning model MDL2, and the like, which will be described later. MDL is simply symbols representing an abbreviation of MODEL.

The processing circuitry 120 includes, for example, an acquisition function 121, an estimation function 122, a reward calculation function 123, a learning function 124, an intervention determination function 125, and an output control function 126. The processing circuitry 120 realizes these functions by, for example, a hardware processor (computer) executing a program stored in the memory 114 (storage circuit). The acquisition function 121 is an example of an “acquisition unit,” the estimation function 122 is an example of an “estimation unit,” the reward calculation function 123 is an example of a “calculation unit,” the learning function 124 is an example of a “learning unit,” the intervention determination function 125 is an example of a “determination unit,” and the output control function 126 is an example of an “output control unit.”

The hardware processor in the processing circuitry 120 is, for example, circuitry such as a central processing unit (CPU), a graphics processing unit (GPU), an application specific integrated circuit (ASIC), a programmable logic device (for example, a simple programmable logic device (SPLD) or a complex programmable logic device (CPLD)), a field programmable gate array (FPGA), or the like. Instead of storing the program in the memory 114, the program may be directly incorporated into the circuitry of the hardware processor. In this case, the hardware processor realizes the functions by reading and executing the program incorporated into the circuitry. The aforementioned program may be stored in advance in the memory 114 or stored in a non-transitory storage medium such as a DVD or a CD-ROM and installed in the memory 114 from the non-transitory storage medium when the non-transitory storage medium is set in a drive device (not shown) of the medical information processing device 100. The hardware processor is not limited to being configured as single circuitry, and may be configured as one hardware processor by combining a plurality of independent circuits to realize each function. Further, a plurality of components may be integrated into one hardware processor to realize each function.

### [Processing Flow of Medical Information Processing Device: Training]

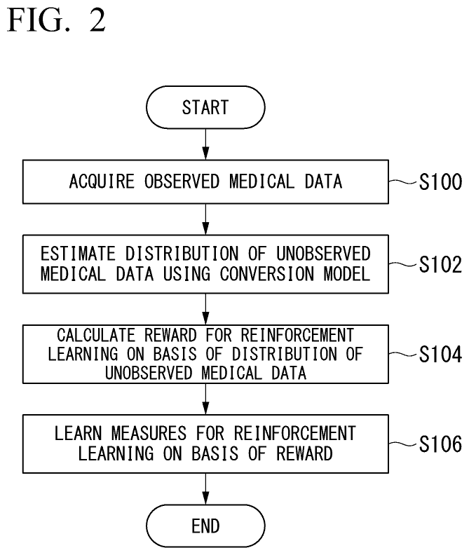

Hereinafter, a series of processing performed by the processing circuitry 120 of the medical information processing device 100 will be described based on a flowchart. FIG. 2 is a flowchart showing a series of processing flows of the processing circuitry 120 according to an embodiment. The processing in this flowchart is executed at the time of training the reinforcement learning model MDL2 which will be described later.

First, the acquisition function 121 acquires data (hereinafter referred to as observed medical data) indicating states of a patient observed before and after intervention (performance) of a medical procedure regarding a predetermined disease (step S100).

The observed medical data includes data obtained by observing the state of the patient before intervention of a medical procedure for the predetermined disease (hereinafter referred to as pre-intervention medical data), and data obtained by observing the state of the patient after intervention of the medical procedure (hereinafter referred to as post-intervention medical data). The medical procedure regarding the predetermined disease is, for example, an examination for the predetermined disease or a treatment for the predetermined disease. The pre-intervention medical data is an example of “first data” and the post-intervention medical data is an example of “second data.”

For example, it can be assumed that various examinations such as for blood pressure, ejection fraction (EF), cardio thoracic ratio (CTR), and cancer markers are performed during a medical procedure. In such a case, the pre-intervention medical data and the post-intervention medical data typically include examination values measured by each examination. For example, if e1 is a blood pressure value, e2 is an EF value, e3 is a CTR value, and e4 is a cancer marker value, the pre-intervention medical data and the post-intervention medical data can be represented as a multidimensional vector such as (e1, e2, e3, e4, . . . ).

Examinations performed before the intervention of a medical procedure may include examinations that are not performed after the intervention of a medical procedure. Similarly, examinations performed after the intervention of a medical procedure may include examinations that are not performed before the intervention of a medical procedure.

For example, among a total of four types of examinations: blood pressure, EF, CTR, and cancer markers, only blood pressure and cancer markers may be examined before intervention of medical procedure, and only EF, CTR, and cancer markers may be examined after intervention of medical procedure. Among the elements of the multidimensional vector indicating the pre-intervention medical data and the post-intervention medical data, arbitrary numerical values or expressions such as null, which indicate that no examinations have been performed, may be stored as elements of examination values for which no examinations have been performed. For example, a multidimensional vector indicating the pre-intervention medical data can be represented as (80, NULL, NULL, 50, . . . ), and a multidimensional vector indicating the post-intervention medical data can be represented as (NULL, 34, 62, 50, . . . ).

In addition to or in place of blood pressure, EF, CTR, and cancer marker values, examination values included in the pre-intervention medical data and the post-intervention medical data may include indices whose numerical values themselves have variations (for example, variance), such as heart disease risk, lung cancer risk, and mortality risk.

For example, the acquisition function 121 may access a database, which is an external device, via the communication interface 111, and acquire observed medical data (pre-intervention medical data and post-intervention medical data) from the database. Furthermore, when a patient's attending physician or the like inputs observed medical data into the input interface 112, the acquisition function 121 may acquire the observed medical data from the input interface 112. Furthermore, if the observed medical data is stored in the memory 114, the acquisition function 121 may acquire the observed medical data from the memory 114.

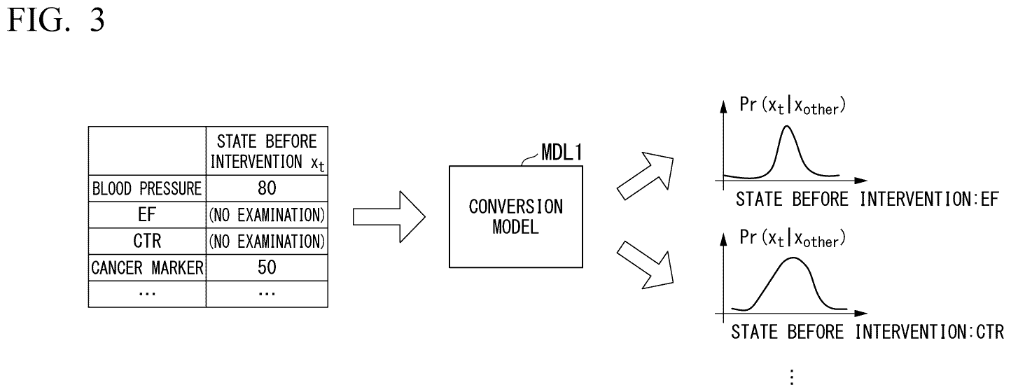

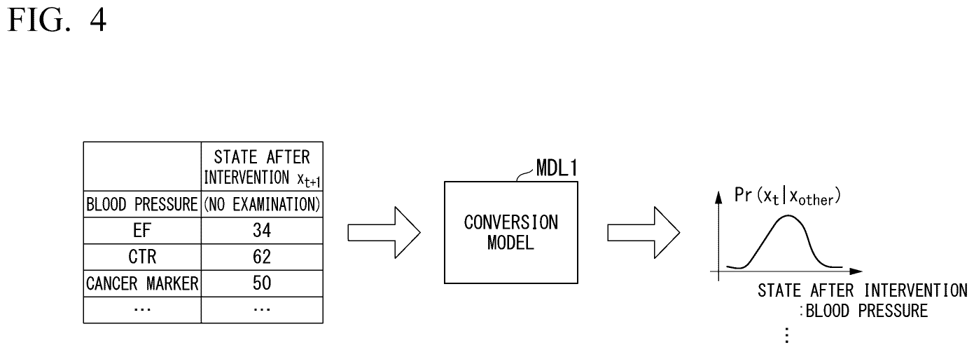

Next, the estimation function 122 estimates a probability density function of data indicating a state of the patient that has not been observed before and after intervention of medical procedure regarding the predetermined disease (hereinafter referred to as unobserved medical data) using the conversion model MDL1 (step S102). FIG. 3 and FIG. 4 are diagrams showing a method of estimating the probability density function of unobserved medical data. In the illustrated example, the time before intervention of medical procedure is t, and the time after intervention of medical procedure is t+1. xₜ represents pre-intervention medical data (or a multidimensional vector representing the pre-intervention medical data), and xₜ₊₁ represents post-intervention medical data (or a multidimensional vector representing the post-intervention medical data).

For example, if at least one of a plurality of predetermined examination values such as blood pressure, EF, CTR, and cancer markers is not present in the pre-intervention medical data xₜ or the post-intervention medical data xₜ₊₁, the estimation function 122 estimates the examination value that is not present in the pre-intervention medical data xₜ or the post-intervention medical data xₜ₊₁ (hereinafter referred to as a missing examination value) and a probability density function of the missing examination value as unobserved medical data.

For example, when blood pressure and cancer markers have been examined and EF and CTR have not been examined at the time t before intervention of medical procedure, the multidimensional vector indicating the pre-intervention medical data xₜ is (80, NULL, NULL, 50, . . . ). When such pre-intervention medical data xx is acquired, the estimation function 122 determines the EF value and CTR value, which are NULL examination values, as missing examination values, and estimates probability density functions of this EF value and CTR value using the conversion model MDL1. The EF value and the CTR value determined as missing examination values that are not present in the pre-intervention medical data xₜ are examples of “first missing examination values,” and the blood pressure value and cancer marker value present in the pre-intervention medical data xₜ are examples of “first existing examination values.”

The conversion model MDL1 may be implemented by a machine learning model, a statistical model, a rule-based model, or a combination thereof. The machine learning model may be, for example, a neural network, a support vector machine, a decision tree, a naive Bayes classifier, a random forest, or the like.

For example, the pre-intervention medical data xₜ is input to the conversion model MDL1. In response to this, the conversion model MDL1 estimates the probability density functions of the EF value and the CTR value of the patient at time t before intervention of the medical procedure from the blood pressure and cancer marker examination values included in the pre-intervention medical data xₜ and outputs the probability density functions.

Missing examination values of the post-intervention medical data xₜ₊₁ are similarly estimated. For example, when EF, CTR, and cancer markers have been examined but blood pressure has not been examined at time t+1 after intervention of the medical procedure, the multidimensional vector indicating the post-intervention medical data xₜ₊₁ is (NULL, 34, 62, 50, . . . ). When such post-intervention medical data xₜ₊₁ is acquired, the estimation function 122 determines the blood pressure value, which is a NULL examination value, as a missing examination value and estimates a probability density function of the blood pressure value using the conversion model MDL1. The blood pressure value determined as a missing examination value that is not present in the post-intervention medical data xₜ₊₁ is an example of a “second missing examination value,” and the EF value, CTR value, and cancer marker value present in the post-intervention medical data xₜ₊₁ are examples of “second existing examination values.”

The post-intervention medical data xₜ₊₁ is input to the conversion model MDL1. In response to this, the conversion model MDL1 estimates the probability density function of the blood pressure value of the patient at time t+1 after intervention of the medical procedure from the examination values of EF, CTR, and cancer markers included in the post-intervention medical data xₜ₊₁ and outputs the probability density function.

Further, the pre-intervention medical data xₜ and the post-intervention medical data xₜ₊₁ may be input to the conversion model MDL1. In this case, the conversion model MDL1 estimates the probability density functions of the EF value and the CTR value of the patient at time t before intervention of the medical procedure from the EF and CTR examination values included in the post-intervention medical data xₜ₊₁ in addition to or in place of the blood pressure and cancer marker examination values included in the pre-intervention medical data xₜ and outputs the probability density functions. In addition, the conversion model MDL1 estimates the probability density function of the blood pressure value of the patient at time t+1 after intervention of the medical procedure from the blood pressure examination value included in the pre-intervention medical data xₜ in addition to or in place of the EF, CTR, and cancer marker examination values included in the post-intervention medical data xₜ₊₁ and outputs the probability density function.

Return to description of the flowchart. Next, the reward calculation function 123 calculates a reward for reinforcement learning on the basis of at least variation in the pre-intervention medical data Xx and variation in the post-intervention medical data xₜ+1 (step S104). “Variation” in each piece of data may be read as “uncertainty.”

“At least” means that, in addition to variation in the pre-intervention medical data xₜ and variation in the post-intervention medical data xₜ₊₁, other elements (one or both of the pre-intervention medical data xₜ and the post-intervention medical data xₜ+1) are included.

Prior to calculating the reward, the reward calculation function 123 first calculates variation in each piece of data (specifically, variation in the probability density function of each piece of data).

For example, the reward calculation function 123 obtains N sampling values by sampling the probability density functions of the pre-intervention medical data xₜ and the post-intervention medical data xₜ₊₁ N times, and calculates a patient's state before intervention sₜ and a patient's state after intervention sₜ₊₁ on the basis of the N sampling values. Then, the reward calculation function 123 calculates variances var(sₜ) and var(sₜ₊₁) thereof as variations. N is any natural number.

Further, instead of the variances, the reward calculation function 123 may calculate other statistical indicators representing variations, such as standard deviation, mean square error, and the average of maximum and minimum values, on the basis of a probability density function.

When the variations in the state before intervention sₜ and the state after intervention sₜ₊₁ are calculated, the reward calculation function 123 calculates a reward on the basis of these variations. For example, the reward may be calculated on the basis of expression (1).

$$\\ \begin{matrix}[ Expression . 1 ] & \\ r = ( {s}_{t + 1} - {s}_{t} ) + ( {u}_{t} - {u}_{t + 1} ) & ( 1 )\end{matrix}$$

r represents the reward, sₜ represents the state before intervention, and s_{t+!} represents the state after intervention. Further, uₜ represents the variation in the state before intervention, and uₜ₊₁ represents the variation in the state after intervention. In other words, sₜ represents the state of the patient at time t before intervention of the medical procedure, sₜ₊₁ represents the state of the patient at time t+1 after intervention of the medical procedure, and uₜ represents the variation in the state of the patient at time t before intervention of the medical procedure and uₜ₊₁ represents the variation in the state of the patient at time t+1 after intervention of the medical procedure.

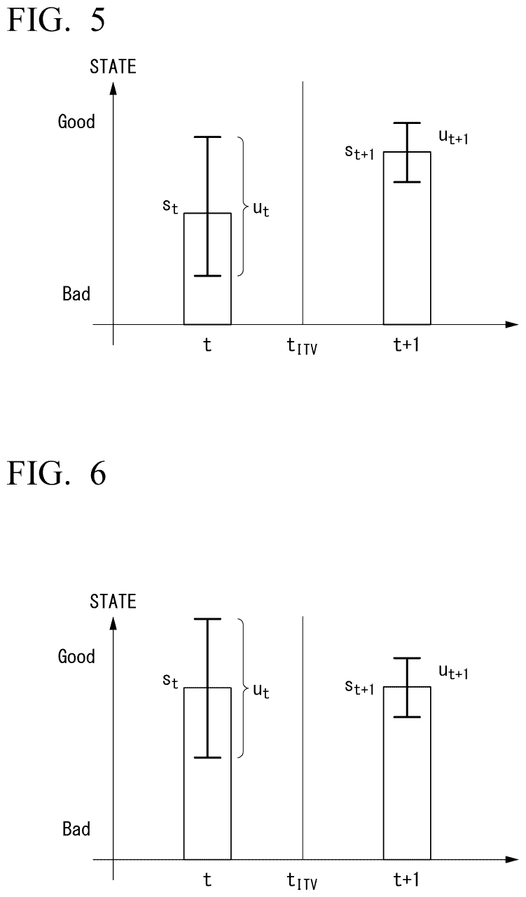

FIG. 5 is a diagram comparing medical data before and after intervention of treatment, which is a medical procedure. For example, as shown in FIG. 5, when comparing medical data before and after intervention of treatment, the state of the patient has improved (s<sₜ₊₁), and furthermore, the variation in the state has decreased (uₜ<uₜ₊₁). In such a case, the first term (sₜ₊₁−sₜ) of expression (1) becomes large, and the second term (uₜ−uₜ₊₁) also becomes large. That is, if the treatment improves the state of the patient and further reduces the variation in the state, the reward r increases.

FIG. 6 is a diagram comparing medical data before and after the intervention of examination, which is a medical procedure. For example, as shown in FIG. 6, when comparing medical data before and after intervention of examination, although the state of the patient is not changed (sₜ≈sₜ₊₁), the variation in the state has decreased (uₜ<uₜ₊₁). In such a case, at least the second term (uₜ−uₜ₊₁) of expression (1) becomes large. That is, if the examination reduces the variation in the state of the patient, the reward r increases.

Further, the reward r may be calculated on the basis of expression (2), for example.

$$\\ \begin{matrix}[ Expression . 2 ] & \\ r = {s}_{t + 1} + ( {u}_{t} - {u}_{t + 1} ) & ( 2 )\end{matrix}$$

As represented by expression (2), the reward calculation function 123 may calculate a larger reward r as the patient' state sₜ₊₁ at time t+1 after intervention of the medical procedure becomes better and the variation (uₜ uₜ₊₁) in the patient's state after intervention of the medical procedure becomes smaller than that before intervention of the medical procedure.

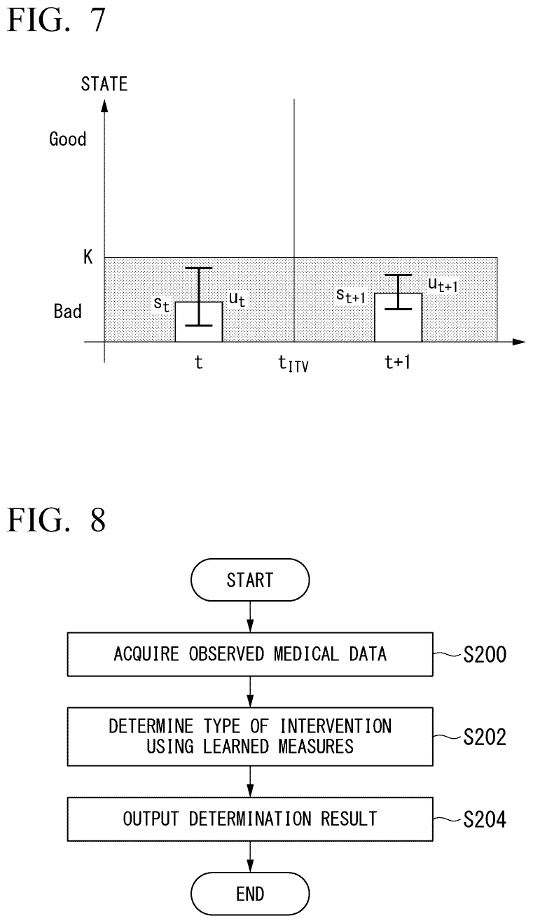

Further, the reward r may be calculated on the basis of expression (3), for example. FIG. 7 is a diagram comparing medical data before and after intervention of treatment which is a medical procedure.

$$\\ \begin{matrix}[ Expression . 3 ] & \\ r = \{ \begin{matrix}{u}_{t} - {u}_{t + 1} & if {s}_{t} , {s}_{t + 1} \leq K ( K is an integer ) \\ ( {s}_{t + 1} - {s}_{t} ) + ( {u}_{t} - {u}_{t + 1} ) & otherwise\end{matrix} & ( 3 )\end{matrix}$$

As shown in expression (3) and FIG. 7, if the patient's state sₜ at time t before intervention or treatment or the patient's state sₜ₊₁ at time t+1 after intervention of treatment is equal to or less than a threshold value K, the reward calculation function 123 may calculate the reward r using only (uₜ−uₜ₊₁). In other words, if the state of the patient does not improve enough to exceed the threshold value K even after treatment, the treatment is considered ineffective, and the reward r may be calculated on the basis of the difference (uₜ−uₜ₊₁) in variations in the state of the patient before and after intervention of the treatment.

Further, the reward r may be calculated on the basis of expression (4), for example.

$$\\ \begin{matrix}[ Expression . 4 ] & \\ r = \frac{( {s}_{t + 1} - {s}_{t} ) + ( {u}_{t} - {u}_{t + 1} )}{C} & ( 4 )\end{matrix}$$

C represents a cost related to a medical procedure. Specifically, C is the number of types of medical procedures and an economic or time cost spent on medical procedures. As represented by expression (4), the reward calculation function 123 may calculate a smaller reward r as the number of types of medical procedures increases (as C increases) or calculate a smaller reward r as the economic or time cost spent on the medical procedures increases (as C increases).

Further, the reward r may be calculated on the basis of expression (5) or expression (6), for example.

$$\\ \begin{matrix}[ Expression . 5 ] & \\ r = {u}_{t} - {u}_{t + 1} & ( 5 )\end{matrix}$$

$$\\ \begin{matrix}[ Expression . 6 ] & \\ r = {s}_{t + 1} - {s}_{t} & ( 6 )\end{matrix}$$

For example, if a medical procedure is either “examination” or “treatment” and the purpose is to reduce variation in the state of the patient (the purpose is to reduce uncertainty), the reward calculation function 123 may calculate the reward using expression (5). Further, if a medical procedure is “treatment” and the purpose is to improve the state of the patient through intervention of the medical procedure, for example, the reward calculation function 123 may calculate the reward r using expression (6). In this manner, the reward calculation function 123 may change the method of calculating the reward r depending on the type of medical procedure for intervention.

Return to description of the flowchart. Next, the learning function 124 leans measures (policies) for the medical procedure on the basis of the calculated reward r (step S106).

For example, the learning function 124 trains the reinforcement learning model MDL2 such that the reinforcement learning function 124 outputs a medical procedure for intervention for a patient that is an observation target whose state is indicated by pre-intervention medical data as measures in response to input of the pre-intervention medical data.

Several types of reinforcement learning are known, such as value-based, policy-based, Actor-Critic, which combines value and policy, and predictive model-based learning. The reinforcement learning model MDL2 according to the present embodiment may be implemented by, for example, a neural network to which value-based Q-learning, Actor-Critic, or the like is applied.

The reinforcement learning model MDL2 is defined by model information stored in the memory 114. The model information includes, for example, various types of information such as connection information on how units included in each of a plurality of layers constituting a neural network are connected to each other, and coupling coefficients assigned to data input and output between connected units. The connection information includes, for example, information such as the number of units included in each layer, information designating the type of unit to which each unit is connected, an activation function that realizes each unit, and a gate provided between units in a hidden layer. The activation function that realizes a unit may be, for example, a normalized linear function (ReLU function), a sigmoid function, a step function, or other functions. The gate selectively passes or weights data transmitted between units depending on a value (e.g., 1 or 0) returned by the activation function. The coupling coefficients include, for example, a weight assigned to output data when data is output from a unit in a certain layer to a unit in a deeper layer in a hidden layer of a neural network. The coupling coefficients may include a bias component specific to each layer, and the like.

For example, when Q learning is applied to the reinforcement learning model MDL2, the learning function 124 may learn an action value function on the basis of expression (7). When Deep Q-Network (DQN), which is a type of Q learning, is applied, the learning function 124 causes the neural network to learn, as an approximation function, an action value function Q(Sₜ, Aₜ) representing a value when a certain medical procedure Aₜ has been intervened under the patient's state sₜ at a certain time t as a function.

$$\\ \begin{matrix}[ Expression . 7 ] & \\ {Q}^{{}_{} ′} ( {S}_{t} , {A}_{t} ) = Q ( {S}_{t} , {A}_{t} ) + α \{ {r}_{t} + γ {max}_{a} Q ( {S}_{t + 1} , a ) - Q ( {S}_{t} , {A}_{t} ) \} & ( 7 )\end{matrix}$$

Q(Sₜ, Aₜ) represents an action value (Q value) at time t before intervention of the medical procedure. α represents a learning rate, and y represents a discount rate. It represents a reward calculated on the basis of expressions (1) to (6). a represents one medical procedure at time t+1 after intervention of medical procedure. The action value is updated on the basis of expression (7) in which the reward n is incorporated in this manner. The reinforcement learning model MDL2 trained in this manner outputs a medical procedure (action variable) Aₜ with the highest value (Q value) among one or a plurality of medical procedures (action variables) Aₜ that can be intervened on the patient at time t before intervention of a medical procedure.

Furthermore, when Actor-Critic is applied to the reinforcement learning model MDL2, for example, the learning function 124 may calculate a gradient on the basis of expression (8) and learn parameters of a value function V_{w} and a measure π_{θ}.

$$\\ \begin{matrix}[ Expression . 8 ] & \\ {∇}_{θ} J ( θ ) = {E}_{τ \sim {π}_{θ}} [ {\sum }_{t = 0}^{T} ( {r}_{t} + γ {V}_{w} ( {S}_{t + 1} ) - {V}_{w} ( {S}_{t} ) ) {∇}_{θ} log {}_{} {π}_{θ} ( {A}_{t} \vert {S}_{t} ) ] & ( 8 )\end{matrix}$$

In addition to Q learning, Actor-Critic, and the like, the learning function 124 may learn a medical procedure for intervention on the patient using reinforcement learning called a bandit algorithm or learn a medical procedure for intervention on the patient using supervised learning, causal inference, or the like instead of such reinforcement learning.

For example, in the case of supervised learning, a machine learning model may be trained on the basis of a training data set in which pre-intervention medical data and the types of medical procedures that can be intervened are used as explanatory variables, and a reward r is used as an objective variable.

For example, in the case of causal inference, the causal inference may be applied using pre-intervention medical data, the type of medical procedure that can be intervened, and the reward r as effects.

The flowchart of FIG. 2 ends according to the series of processing described above.

### [Processing Flow/Runtime of Medical Information Processing Device]

FIG. 8 is a flowchart showing a series of processing flows of the processing circuitry 120 according to an embodiment. The processing of the flowchart shown in FIG. 8 may be executed, for example, after training of the reinforcement learning model MDL2 to which Q learning, Actor-Critic, bandit algorithm, or the like is applied is completed. If measures (i.e., a medical procedure for intervention on the patient) are learned using supervised learning or causal inference instead of reinforcement learning such as Q learning, Actor-Critic, or bandit algorithm, the processing of the flowchart is executed after such supervised learning or causal inference is executed.

First, the acquisition function 121 acquires data indicating a state of a patient who is a target for intervention of a medical procedure (hereinafter referred to as a target patient) (that is, pre-intervention medical data of the target patient) (step S200). The pre-intervention medical data of the target patient is an example of “third data.”

For example, similarly to processing of S102, the acquisition function 121 may access a database, which is an external device, via the communication interface 111, and acquire the pre-intervention medical data of the target patient from the database. Furthermore, if a target patient's attending physician or the like inputs the pre-intervention medical data to the input interface 112, the acquisition function 121 may acquire the pre-intervention medical data of the target patient from the input interface 112. Further, if the pre-intervention medical data of the target patient is stored in the memory 114, the acquisition function 121 may acquire the pre-intervention medical data of the target patient from the memory 114.

Next, the intervention determination function 125 determines a medical procedure (or the type thereof) for intervention on the target patient from the pre-intervention medical data of the target patient using the learned measures (step S202).

For example, as described above, the reinforcement learning model MDL2 is trained to output a medical procedure (action variable) Aₜ with the highest value (Q value) among one or a plurality of medical procedures (action variables) Aₜ that can be intervened on the patient at time t before intervention f a medical procedure. Therefore, the intervention determination function 125 inputs the pre-intervention medical data of the target patient to the trained reinforcement learning model MDL2. In response to input of the pre-intervention medical data of the target patient, the trained reinforcement learning model MDL2 outputs a medical procedure with the highest value (Q value) among one or a plurality of medical procedures that can be intervened on the target patient. The intervention determination function 125 determines the medical procedure output by the reinforcement learning model MDL2 as a medical procedure for intervention on the target patient.

Next, the output control function 126 outputs information based on the medical procedure determined by the intervention determination function 125 via the output interface 113 (step S204). For example, the output control function 126 may cause the display 113a to display the information based on medical procedure. Further, the output control function 126 may transmit the information based on the medical procedure to an external device (for example, a computer used by the target patient's attending physician) via the communication interface 111. Accordingly, the processing of this flowchart ends.

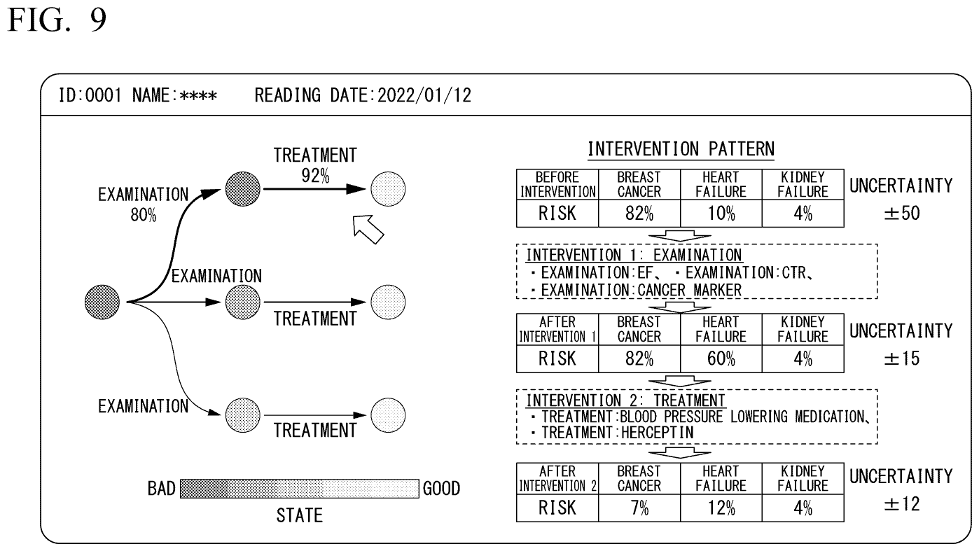

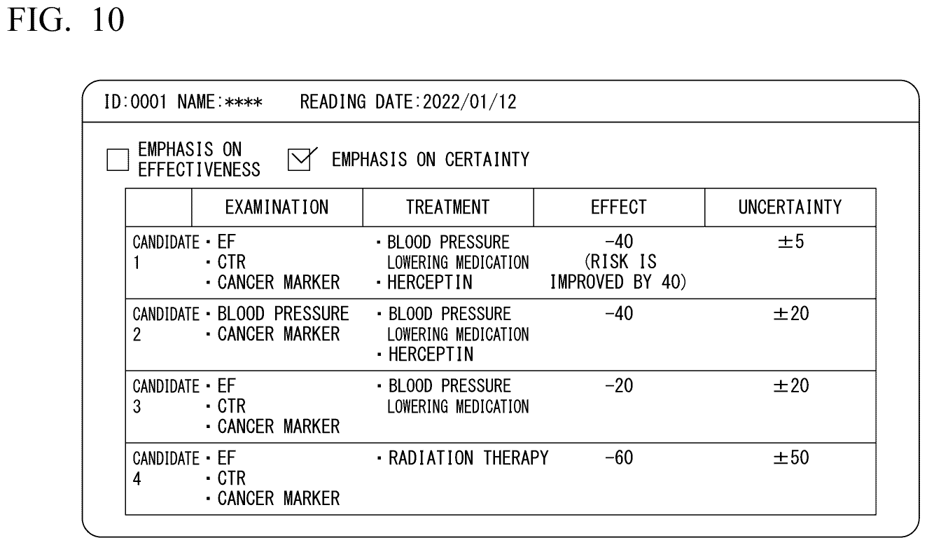

FIG. 9 and FIG. 10 are diagrams showing examples of screens of the display 113a on which information based on medical procedures for intervention on the target patient is displayed. For example, the screen of the display 113a may display the second most valuable medical procedure, the third most valuable medical procedure, and the like in addition to the medical procedure with the highest value (Q value). Furthermore, the screen of the display 113a may display the type (treatment or examination) of medical procedure for intervention on the target patient, or display the uncertainty when each medical procedure has been intervened (i.e. variations in the state of the target patient before and after intervention of a medical procedure) as a quantitative index such as probability. By displaying such information on the display 113a, it is possible to recommend a medical procedure for intervention on the patient.

According to the embodiments described above, the processing circuitry 120 of the medical information processing device 100 acquires pre-intervention medical data indicating a state of a patient before intervention of a medical procedure related to a predetermined disease, and post-intervention medical data indicating a state of the patient after intervention of the medical procedure. The processing circuitry 120 calculates a reward for reinforcement learning on the basis of at least variation in the pre-intervention medical data and variation in the post-intervention medical data. The processing circuitry 120 learns measures for determining a medical procedure for intervention on the patient on the basis of the calculated reward.

By using the measures learned in this manner, it is possible to determine a medical procedure for intervention on the target patient from the pre-intervention medical data of the target patient. As a result, it is possible to recommend a medical procedure for intervention on the target patient.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical information processing device using an artificial intelligence model, comprising processing circuitry configured to:
acquire first data indicating a state of a patient before intervention of a medical procedure related to a predetermined disease, and second data indicating a state of the patient after intervention of the medical procedure;
calculate a reward on the basis of at least a variation in the first data and a variation in the second data;
cause the artificial intelligence model to learn policies for the medical procedure on the basis of the reward; and
update the artificial intelligence model through a learning of the policies.

2. The medical information processing device according to claim 1, wherein the first data and the second data are multidimensional data including a plurality of examination values, and
when at least one examination value among the plurality of examination values is not present in the first data or the second data, the processing circuitry estimates a missing examination value corresponding to the examination value that is not present in the first data or the second data, and a variation in the missing examination value.

3. The medical information processing device according to claim 2, wherein the processing circuitry estimates a first missing examination value corresponding to an examination value that is not present in the first data, and a variation in the first missing examination value using a first existing examination value that is an examination value present in the first data or a second existing examination value that is an examination value present in the second data when at least one examination value among the plurality of examination values is not present in the first data, and estimates a second missing examination value corresponding to an examination value that is not present in the second data, and a variation in the second missing examination value using the second existing examination value or the first existing examination value when at least one examination value among the plurality of examination values is not present in the second data.

4. The medical information processing device according to claim 1, wherein the processing circuitry calculates the reward further on the basis of the state of the patient indicated by the first data and the state of the patient indicated by the second data in addition to the variation in the first data and the variation in the second data.

5. The medical information processing device according to claim 1, wherein the processing circuitry calculates the reward further on the basis of the state of the patient indicated by the second data in addition to the variation in the first data and the variation in the second data.

6. The medical information processing device according to claim 1, wherein the processing circuitry calculates the reward further on the basis of the number of types of medical procedures in addition to the variation in the first data and the variation in the second data.

7. The medical information processing device according to claim 1, wherein the processing circuitry calculates the reward further on the basis of an economic or time cost of the medical procedure in addition to the variation in the first data and the variation in the second data.

8. The medical information processing device according to claim 1, wherein the processing circuitry determines the medical procedure for intervention on a target patient on the basis of third data indicating a state of the target patient and the learned policies, and outputs information based on the determined medical procedure via an output interface.

9. A medical information processing method using a computer, comprising:
acquiring first data indicating a state of a patient before intervention of a medical procedure related to a predetermined disease, and second data indicating a state of the patient after intervention of the medical procedure;
calculating a reward on the basis of at least a variation in the first data and a variation in the second data;
causing an artificial intelligence model to learn policies for the medical procedure on the basis of the reward; and
updating the artificial intelligence model through a learning of the policies.

10. A computer-readable non-transitory storage medium storing a program to be executed by a computer, the program comprising:
acquiring first data indicating a state of a patient before intervention of a medical procedure related to a predetermined disease, and second data indicating a state of the patient after intervention of the medical procedure;
calculating a reward on the basis of at least a variation in the first data and a variation in the second data;
causing an artificial intelligence model to learn policies for the medical procedure on the basis of the reward; and
updating the artificial intelligence model through a learning of the policies.
